(19) United States
(12) Patent Application Publication
(10) Pub. No.: US 2022/0031155 A1
(43) Pub. Date: Feb. 3, 2022

(54) MEDICAL OBSERVATION SYSTEM, MEDICAL LIGHT SOURCE APPARATUS, AND MEDICAL ILLUMINATION METHOD

(71) Applicant: Sony Corporation, Tokyo (JP)

(72) Inventors: Tetsuaki IWANE, Tokyo (JP); Yuichi TAKAHASHI, Tokyo (JP)

(73) Assignee: Sony Corporation, Tokyo (JP)

(21) Appl. No.: 17/278,672

(22) PCT Filed: Sep. 19, 2019
(86) PCT No.: PCT/JP2019/036753

§ 371 Date: Mar. 23, 2021

(30) Foreign Application Priority Data
Oct. 1, 2018 (JP) 2018-186804

(51) Int. Cl. A61B 1/06; A61B 1/07

(52) U.S. Cl. CPC A61B 1/063; A61B 1/07

(57) ABSTRACT

A medical observation system according to an embodiment of the present technology includes a light source, an optical member, a first light guide body, and an imaging element. The light source has a plurality of light-emitting elements, each of which emits light. The optical member is arranged to reflect the light emitted from the plurality of light-emitting elements and make the reflected light incident on a first region. The first light guide body is arranged in the first region, has an incident end and an emission end on a side opposite to the incident end, and guides the light incident from the incident end to the emission end. The imaging element irradiates an operating field with the guided light and captures an image of light reflected by a subject.

### TECHNICAL FIELD

The present technology relates to a medical observation system, a medical light source apparatus, and a medical illumination method used in medical observation.

### BACKGROUND ART

Conventionally, the light sources of observation apparatuses for observing living-body tissues such as endoscopic apparatuses and microscopic apparatuses have been developed. Recently, there have been a lot of opportunities to use light-emitting elements such as LDs (Laser Diodes) instead of conventional lamp light sources as the light sources of such medical observation apparatuses.

For example, Patent Literature 1 describes an illumination device used in an observation apparatus that observes operating fields. In the illumination device, three laser light sources that emit light corresponding to light's three primary colors are provided. Laser light emitted from the respective laser light sources is multiplexed together as one light flux by three dichroic mirrors that reflect the light of respective wavelength bands. The multiplexed light flux passes through a diffusion member that integrates the divergence angles of the respective laser light and reduces color unevenness or the like during irradiation. The light flux having passed through the diffusion member is multiplexed with other white light and condensed on a light guide via a condensing lens (paragraphs [0027], [0037], [0039], [0046], and [0077] of the specification, FIG. 1, or the like in Patent Literature 1).

### CITATION LIST

### Patent Literature

Patent Literature 1: Japanese Patent Application Laid-open No. 2016-120104

### DISCLOSURE OF INVENTION

### Technical Problem

In a configuration in which a plurality of light-emitting elements is provided as described above, there is a possibility that an apparatus size increases with an increase in the number of optical systems used in multiplexing of light, condensing, or the like. Therefore, technologies to reduce apparatus sizes and realize excellent observation have been demanded.

In view of the above circumstances, the present technology has an object of providing a medical observation system, a medical light source apparatus, and a medical illumination method that reduce an apparatus size and realize excellent observation.

### Solution to Problem

In order to achieve the above object, a medical observation system according to an embodiment of the present technology includes a light source, an optical member, a first light guide body, and an imaging element.

The light source has a plurality of light-emitting elements, each of which emits light.

The optical member is arranged to reflect the light emitted from the plurality of light-emitting elements and make the reflected light incident on a first region.

The first light guide body is arranged in the first region, has an incident end and an emission end on a side opposite to the incident end, and guides the light incident from the incident end to the emission end.

The imaging element irradiates an operating field with the guided light and captures an image of light reflected by a subject.

In the medical observation system, the light emitted from the plurality of light-emitting elements is reflected by the optical member and incident on the first region. The light incident on the first region is incident on the incident end of the first light guide body arranged in the first region and guided to the emission end. The guided light is irradiated onto the operating field, and the light reflected by the subject is shot. As described above, the reflection of the light makes it possible to shorten a distance for condensing. Further, the light condensed by the light guide body is uniformized as it is. Thus, it is possible to reduce an apparatus size and realize excellent observation.

The first light guide body may uniformize brightness distribution at the emission end of the light emitted from the emission end.

Thus, it is possible to emit light having uniform brightness distribution. As a result, it is possible to perform the irradiation of light having small brightness unevenness and realize excellent observation.

The plurality of light-emitting elements may be arranged around a prescribed axis. In this case, the optical member may have a first reflection unit that is arranged facing the plurality of light-emitting elements and reflects the light emitted from the plurality of light-emitting elements to be condensed toward a second region on the prescribed axis.

Thus, it is possible to fold back light emitted from a plurality of light-emitting elements to be condensed and reduce an apparatus size.

The plurality of light-emitting elements may emit the light parallel to the prescribed axis. Thus, light parallel to each other is emitted from a plurality of light-emitting elements, and it is possible to easily condense light emitted from a plurality of light-emitting elements.

The first reflection unit may include at least one of a parabolic mirror or a free-form surface mirror.

Thus, it is possible to improve, for example, condensing accuracy. As a result, it is possible to perform the irradiation of bright light with an improvement in light condensing efficiency and realize excellent observation.

The free-form surface mirror may include a plurality of divided mirrors.

For example, the adjustment of the angles or the like of respective divided mirrors makes it possible to sufficiently improve condensing accuracy. Further, the use of divided mirrors makes it possible to reduce an apparatus size.

The second region may be the first region. Thus, the light reflected by the first reflection unit is directly condensed on the first light guide body. As a result, it is possible to reduce the number of parts and reduce a manufacturing cost.

The optical member may have a second reflection unit that is arranged facing the first reflection unit and reflects the light toward the first region, the light being directed from the first reflection unit to the second region.

For example, the adjustment of the second reflection unit makes it possible to improve condensing efficiency. As a result, it is possible to perform the irradiation of bright light and realize excellent observation.

The second reflection unit may include at least one of a parabolic mirror, a plane mirror, or a free-form surface mirror.

Thus, it is possible to sufficiently improve condensing accuracy and easily obtain a bright observation image or the like.

The plurality of light-emitting elements may include a plurality of types of light-emitting elements that emits light of different wavelength ranges.

Thus, it is possible to easily adjust the color or the like of light irradiated onto an observation portion and obtain a high-quality observation image or the like. As a result, it is possible to realize excellent observation.

The plurality of light-emitting elements may include at least one of a light-emitting element that emits red light, a light-emitting element that emits green light, or a light-emitting element that emits blue light.

Thus, it is possible to emit white light. For example, the control of the outputs of respective groups makes it possible to adjust the color of the white light and obtain a sufficiently high-quality observation image or the like.

The plurality of light-emitting elements may include at least one of a light-emitting element that emits infrared light or a light-emitting element that emits ultraviolet light.

Thus, it is possible to emit, for example, excitation light or the like that excites a fluorescent body. As a result, it is possible to perform the fluorescent observation or the like of an observation portion and realize detailed observation.

The plurality of light-emitting elements may be arranged such that an incident angle of the light with respect to the incident end falls within a prescribed range, the light being emitted from at least one of the same type of light-emitting elements.

Thus, it is possible to alleviate, for example, the deviations or the like of beam shapes when the light of respective wavelength ranges is condensed. As a result, it is possible to sufficiently uniformize brightness distribution or the like at the emission end.

The plurality of light-emitting elements may include laser diodes.

Thus, it is possible to condense light on, for example, a thin light guide body at high condensing efficiency and realize an observation apparatus or the like that has low invasiveness and allows bright observation.

The plurality of light-emitting elements may be arranged on the same radiation plate.

Thus, it is possible to easily cool the respective light-emitting elements. As a result, it is possible to easily improve the reliability of an apparatus.

The medical observation system may further include: a second light guide body that guides the light to an observation target; and a relay optical system that connects the light emitted from the emission end of the first light guide body to an incident end of the second light guide body.

Thus, it is possible to properly guide the light uniformized by the first light guide body to an observation portion. As a result, it is possible to excellently observe an observation portion.

An area of the emission end of the first light guide body may be smaller than an area of the incident end of the second light guide body.

Thus, it is possible to efficiently guide the light uniformized by the first light guide body. As a result, it is possible to perform the irradiation of light that is bright and have no brightness unevenness and observe an observation portion.

The medical observation system may be constituted as a microscopic system or an endoscopic system.

Thus, it is possible to properly observe an operating field or the like of a patient.

A medical light source apparatus according to an embodiment of the present technology includes a light source, an optical member, and a light guide body.

The light source has a plurality of light-emitting elements, each of which emits light.

The optical member is arranged to reflect the light emitted from the plurality of light-emitting elements and make the reflected light incident on a prescribed region.

The light guide body that is arranged in the prescribed region, has an incident end and an emission end on a side opposite to the incident end, and guides the light incident from the incident end to the emission end.

A medical illumination method according to an embodiment of the present technology includes causing each of a plurality of light-emitting elements to emit light.

The light emitted from the plurality of light-emitting elements is reflected, and the reflected light is made incident on a prescribed region.

The light incident from the incident end is guided to the emission end by a light guide body that is arranged in the prescribed region and has the incident end and an emission end on a side opposite to the incident end.

### Advantageous Effects of Invention

According to the present technology, it is possible to reduce an apparatus size and realize excellent observation as described above. Note that the effect described here should not be interpreted in a limited way, and any effect described in the present disclosure may be produced.

### BRIEF DESCRIPTION OF DRAWINGS

FIG. 1 is a schematic view showing a configuration example of a medical observation system according to an embodiment of the present technology.

FIGS. 2A and 2B are schematic views each showing an example of the arrangement of laser diodes.

FIGS. 3A and 3B are schematic views each showing an example of the brightness distribution of an end surface of an inner light guide.

FIG. 4 is a schematic view showing another configuration example of a light source unit.

FIG. 5 is a schematic view showing another configuration example of the light source unit.

FIG. 6 is a schematic view showing another configuration example of the light source unit.

FIG. 7 is a schematic view showing another configuration example of the light source unit.

FIG. 8 is a schematic view showing another configuration example of the light source unit.

FIG. 9 is a view depicting an example of a schematic configuration of an endoscopic surgery system according to another embodiment.

FIG. 10 is a view depicting an example of a schematic configuration of a microscopic surgery system according to another embodiment.

### MODE(S) FOR CARRYING OUT THE INVENTION

Hereinafter, embodiments according to the present technology will be described with reference to the drawings.

[Configuration of Medical Observation System]

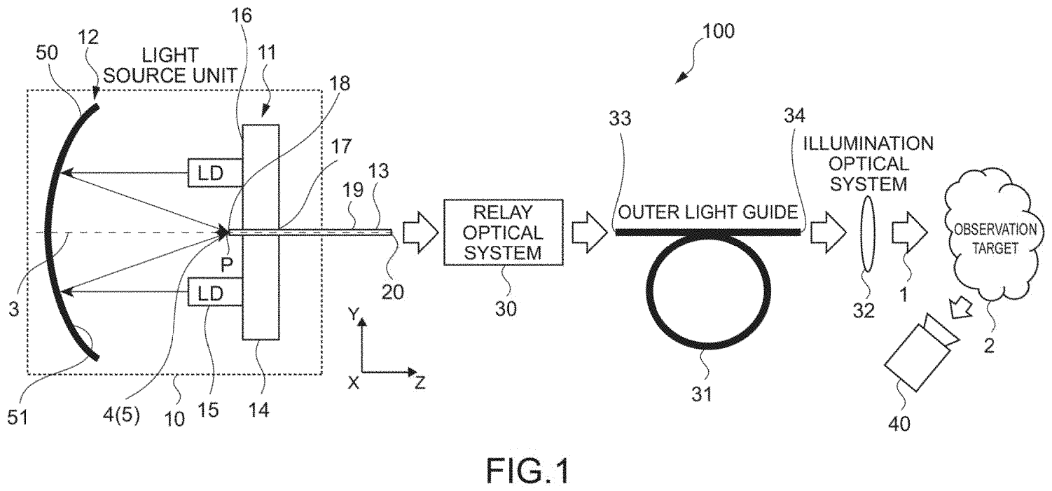

FIG. 1 is a schematic view showing a configuration example of a medical observation system according to an embodiment of the present technology. The medical observation system 100 is constituted as, for example, an observation system such as a microscopic system and an endoscopic system for observing an affected part or the like of a patient. Light (hereinafter described as irradiation light 1) emitted from the medical observation system 100 is irradiated onto an operating field such as an affected part or the like of a patient that is an observation target 2 of an operator. Note that an operating field in the present disclosure includes, besides a target region in a medical action such as a surgical operation, an observation visual field or the like for observing living-body tissues. For example, when the observation target 2 onto which the irradiation light 1 has been irradiated is shot as a subject by an imaging element 40 or the like, the state of the observation target 2 is observed.

The medical observation system 100 has a light source unit 10, a relay optical system 30, an outer light guide 31, an illumination optical system 32, and the imaging element 40.

The light source unit 10 generates light that serves as the irradiation light 1 and emits the generated light along a light axis 3. In FIG. 1, a sectional view of the light source unit 10 cut along a surface including the light axis 3 is schematically shown. Hereinafter, a side on which the light that serves as the irradiation light 1 is emitted will be described as the front side of the light source unit 10, and its opposite side will be described as the rear side of the light source unit 10.

Further, a direction in which the light axis 3 extends will be described as the longitudinal direction (Z direction) of the light source unit 10, and a direction perpendicular to the section (space) of FIG. 1 will be described as the horizontal (X direction) of the light source unit 10. Further, a direction (up-and-down direction in the figure) perpendicular to the longitudinal direction and the horizontal direction will be described as the vertical direction (Y direction) of the light source unit 10.

The light source unit 10 has a light source 11, an optical member 12, and an inner light guide 13. In the present embodiment, the light source unit 10 corresponds to a medical light source apparatus, and the light axis 3 corresponds to a prescribed axis. Further, a medical illumination method according to the present embodiment is realized by the light source unit 10.

The light source 11 has a radiation unit 14 and a plurality of laser diodes (LD) 15. In the present embodiment, the radiation unit 14 corresponds to a radiation plate, and the plurality of laser diodes 15 corresponds to a plurality of light-emitting elements.

The radiation unit 14 is a member that radiates heat generated by the plurality of laser diodes 15. The radiation unit 14 has a flat plate shape of which the plane shape is a square and has an arrangement surface 16 on its one surface on which the plurality of laser diodes 15 is arranged. In other words, the radiation unit 14 function also as a support member that supports the laser diodes 15.

The arrangement surface 16 has a square opening part 17 at its central area. Further, the radiation unit 14 is arranged to be orthogonal to the light axis 3 at the center (the center of the opening part 17) of the arrangement surface 16. Note that the arrangement surface 16 is a surface on the rear side of the radiation unit 14 (the light source unit 10).

The radiation unit 14 includes, for example, a heat conductivity material having relatively high heat conductivity such as copper, aluminum, a graphite sheet, and nitride aluminum. A specific configuration of the radiation unit 14 is not limited. For example, a resin substrate such as an epoxy substrate and a plastic substrate and a heat conductivity material may be combined together to constitute the radiation unit 14. Further, the radiation unit 14 may have a radiation fin (heat sink) or the like on its surface on a side opposite to the arrangement surface 16.

Each of the plurality of laser diodes 15 is a light-emitting element that emits laser light. The respective laser diodes 15 are arranged on the arrangement surface 16 of the radiation unit 14. As described above, the plurality of laser diodes 15 is arranged on the same radiation unit 14. Thus, it is possible to efficiently cool the respective laser diodes 15.

Note that an emission side on which laser light is emitted is directed to a side opposite to the radiation unit 14 (the arrangement surface 16), that is, the rear side of the light source unit 10. Accordingly, the respective laser diodes 15 emit the laser light toward the rear side of the light source unit 10.

In the present embodiment, the plurality of laser diodes 15 emits the laser light parallel to the light axis 3. That is, a plurality of the laser light parallel to each other is emitted toward the rear side of the light source unit 10 from the arrangement surface 16. Note that parallel in the present disclosure includes a substantially parallel state. For example, the laser light emitted within an angle range in which it is possible to properly condense the laser light with the optical member 12 that will be described later is included in the laser light parallel to each other.

In an example shown in FIG. 1, two laser diodes 15 that emit the laser light toward the rear side of the light source unit 10 are schematically shown. Of course, the number of the laser diodes 15 is not limited but may be appropriately selected according to, for example, the purpose or the like of the medical observation system 100 or in order to allow the realization of a desired light amount (brightness).

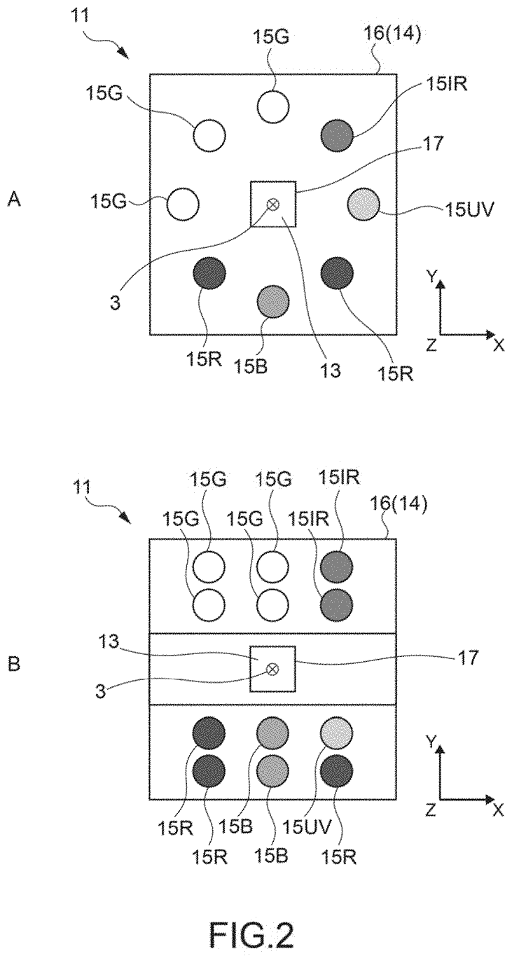

FIGS. 2A and 2B are schematic views each showing an example of the laser diodes 15. In FIGS. 2A and 2B, the arrangement surface 16 (the radiation unit 14) when seen from the rear side of the light source unit 10 is schematically shown. Note that the inner light guide 13 that will be described later is arranged in the square opening part 17 at the center.

In an example shown in FIG. 2A, eight laser diodes 15 are concentrically arranged about the light axis 3. The concentric arrangement of the laser diodes 15 like this makes it possible to uniformize the characteristics (such as incident angles and reflection angles with respect to respective parts) of light paths through which respective laser light passes. This point will be described in detail later.

In the present embodiment, a plurality of types of laser diodes 15 that emits the light of different wavelength ranges is used as the plurality of laser diodes 15. In FIG. 2A, different types of the laser diodes 15 are shown by different colors. The respective laser diodes 15 are driven independently of each other by a controller or the like not shown. That is, it is possible to control the outputs of the laser light of different wavelength ranges independently of each other.

In the present embodiment, laser diodes 15R that emit red light, laser diodes 15G that emit green light, and a laser diode 15B that emits blue light are used. The use of the laser diodes 15R to 15B that emit the respective colors of the light of RGB representing light's three primary colors like this makes it possible to generate white light. Thus, it is possible to irradiate the observation target 2 with the white light (the irradiation light 1) to perform the visible-light observation or the like of the observation target 2.

As the laser diodes 15R that emit red light, GaInP quantum well structure laser diodes or the like are, for example, used. Further, as the laser diodes 15G that emit green light, GaInN quantum well structure laser diodes or the like are, for example, used. Further, as the laser diode 15B that emits blue light, a GaInN quantum well structure laser diode or the like is, for example, used. Besides this, arbitrary laser diodes 15 capable of emitting red light, green light, and blue light may be used.

Further, in the present embodiment, a laser diode 15IR that emits infrared light and a laser diode 15UV that emits ultraviolet light are used. For example, the irradiation of the observation target 2 with infrared light makes it possible to shoot an infrared image or the like of the observation target 2 and observe not only the surface but also the inner state or the like of the observation target 2 in detail.

Further, for example, the use of ultraviolet light makes it possible to excite a highlighter or the like. Thus, it is possible to detect fluorescence emitted from the highlighter or the like and easily identify a lesion part. Note that such fluorescence imaging is made possible by light (the respective single colors of RGB, infrared light, or the like) other than ultraviolet light according to the type of the highlighter or the like.

As the laser diode 15IR that emits infrared light, a GaAlAs-based or GaAs-based laser diode or the like is, for example, used. Further, as the laser diode 15UV that emits ultraviolet light, a GaN-based laser diode or the like is, for example, used. Besides this, arbitrary laser diodes 15 that emit the light of an invisible region such as infrared light and ultraviolet light may be used.

In the example shown in FIG. 2A, the two laser diodes 15R for red light and the three laser diodes 15G for green light are arranged. Further, each of the laser diode 15B for blue light, the laser diode 15IR for infrared light, and the laser diode 15UV for ultraviolet light is singly arranged. The numbers or the like of the provided laser diodes 15 of the respective colors (wavelength ranges) are not limited. For example, in order to allow the realization of the intensity, color, or the like of the desired irradiation light 1, the numbers or the like of the various laser diodes 15 may be set. Further, the numbers or the like of the used laser diodes 15 may be set according to, for example, the output characteristics or the like of the respective laser diodes 15.

In an example shown in FIG. 2B, 12 laser diodes 15 are arranged in a lattice pattern with respect to the light axis 3. Specifically, six respective laser diodes 15 are arranged in one region (an upper side in the figure) and the other region (a lower side in the figure) of the arrangement surface 16 across the light axis 3 (the opening part 17). In the respective regions, the six laser diodes 15 are arranged in a two-by-three lattice pattern so that three out of the six laser diodes 15 are arranged side by side in the horizontal direction (X direction), and that two out of the six laser diodes 15 are arranged side by side in the vertical direction (Y direction). Hereinafter, the arrangement of the respective laser diodes 15 will be described assuming that the lower-left arrangement position (X, Y) in the figure is (1, 1). In this case, the upper right arrangement position (X, Y) is (3, 4).

In the lower region, three laser diodes 15R for red light, two laser diodes 15B for blue light, and one laser diode 15UV for ultraviolet light are arranged. The laser diodes 15R are arranged at the arrangement positions (1, 1), (1, 2), and (3, 1).

Further, the laser diodes 15B are arranged at the arrangement positions (2, 1) and (2, 2). Further, the laser diode 15UV is arranged at the arrangement position (3, 2).

In the upper region, four laser diodes 15G for green light and two laser diodes 15IR for infrared light are arranged. The laser diodes 15G are arranged at the arrangement positions (1, 3), (1, 4), (2, 3), and (2, 4). Further, the laser diodes 15IR are arranged at the arrangement positions (3, 3) and (3, 4).

In this arrangement, at least one of the respective colors of the laser diodes 15 is arranged at outer arrangement positions (arrangement positions other than the inner arrangement positions (2, 2) and (2, 3)). Thus, it is possible to uniformize the characteristics of light paths through which the respective laser light passes.

The lattice arrangement of the laser diodes 15 makes it possible to easily perform, for example, the dense arrangement of the respective laser diodes 15. Thus, it is possible to increase the number of the mountable laser diodes 15 and improve output intensity (the brightness of the irradiation light 1) or the like. Further, the arrangement of the laser diodes 15 in divided regions is facilitated. Thus, it is possible to perform, for example, a reduction in the use amount of a heat conductive material or the like used in the radiation unit 14 and the weight reduction of the apparatus.

In the present embodiment, the plurality of laser diodes 15 is arranged around the light axis 3 as described above. The three-dimensional arrangement of the laser diodes 15 around the light axis 3 makes it possible to easily constitute, for example, optical systems or the like symmetric with respect to the light axis 3 and easily arrange a multiplicity of the laser diodes 15. Further, as will be described later, the control of the light paths of the respective laser light with common optical systems and the simplification of a configuration are also made possible.

Note that the arrangement examples described with reference to FIGS. 2A and 2B are given only as examples, and the present technology is not limited to the arrangement examples. That is, the arrangement positions, the numbers, or the like of the respective laser diodes 15 may be appropriately set. Arbitrary arrangement may be employed according to, for example, the types or the numbers of the used laser diodes 15 or the desirable size, functions, or the like of the light source unit 10.

Referring back to FIG. 1, the optical member 12 is arranged to reflect the light emitted from the plurality of laser diodes 15 and make the reflected light incident on a condensing region 4. In other words, the optical member 12 reflects the respective laser light to be put together in the condensing region 4. Note that the condensing region 4 is, for example, a condensing spot in which the respective laser light is put together. In the present embodiment, the condensing region 4 is set at a prescribed position on the light axis 3 as a region on a plane (XY plane) orthogonal to the longitudinal direction (Z direction) of the light source unit 10. In the present embodiment, the condensing region 4 corresponds to a first region.

The optical member 12 has a reflector 50. The reflector 50 is a parabolic mirror and has a recessed reflection surface 51. Note that in the present disclosure, the parabolic mirror is a mirror (reflector) in which at least a partial sectional shape of the reflection mirror includes a parabola.

In the present embodiment, the reflection surface 51 includes a recessed rotation paraboloid obtained by rotating a prescribed parabola with the axis of the parabola as a central axis. That is, the reflector 50 is a rotationally-symmetric parabolic mirror with a recessed paraboloid as the reflection surface 51.

The reflector 50 is arranged to make the central axis of the reflection surface 51 coincident with the light axis 3 with the reflection surface 51 directed to the arrangement surface 16 (the emission side of the plurality of laser diodes 15) of the light source 11. Accordingly, as shown in FIG. 1, a sectional shape including the light axis 3 of the reflection surface 51 is a parabolic shape that opens toward the side of the arrangement surface 16 (the front side of the light source unit 10).

In the present embodiment, the laser light is emitted parallel to the light axis 3 from the respective laser diodes 15 as described above. That is, the laser light parallel to the central axis (light axis 3) is incident on the reflection surface 51. In FIG. 1, the light paths of the laser light in a section (YZ plane) including the light axis 3 are schematically shown by arrows.

The laser light incident parallel to the central axis is reflected toward a focus P (a focus P of the parabola constituting the section) of the reflection surface 51 that is a rotation paraboloid. In other words, the respective laser light is reflected by the reflection surface 51 and condensed toward the focus P. Note that the focus P of the reflection surface 51 is a point on the light axis 3.

Further, the respective laser light is condensed at a finite spot size at the focus P. That is, at the focus P, a state in which the respective laser light is condensed in a constant region is realized. Hereinafter, a region (spot) in which the respective laser light is condensed by the reflection surface 51 will be described as a focus region 5.

As described above, the reflector 50 is arranged facing the plurality of laser diodes 15, reflects the laser light emitted from the plurality of laser diodes 15, and condenses the reflected laser light toward the focus region 5 on the light axis 3. In the present embodiment, the reflector 50 corresponds to a first reflection unit, and the focus region 5 corresponds to a second region. Further, the reflector 50 is an example of a reflection plate.

Further, in the present embodiment, the focus region 5 that serves as the focus P of the reflector 50 is the condensing region 4. In other words, the reflector 50 reflects the light emitted from the plurality of laser diodes 15 and condenses the reflected light toward the condensing region 4(the focus region 5) on the XY plane. Note that without being limited to a parabolic mirror, a mirror having an arbitrary shape that is capable of condensing the light in the condensing region 4 may be, for example, used as the reflector 50. A free-form surface mirror or the like may be, for example, used as the reflector 50. The free-form surface mirror is appropriately designed according to, for example, a light-path simulation or the like. Alternatively, the free-form surface mirror may be constituted to correct aberration or the like for condensing the respective laser light. Besides this, the shape of the reflector 50 (the reflection surface 51) is not limited.

A specific configuration of the reflector 50 is not limited. As a material constituting the reflector 50, an arbitrary material such as an acrylic resin, glass, and metal may be, for example, used. By, for example, subjecting these materials to mirror finish to have prescribed surface roughness, the reflector 50 is constituted. Besides this, an arbitrary material may be used according to, for example, processing accuracy, productivity, or the like.

Further, for example, the reflection surface 51 of the reflector 50 may be subjected to high-reflection coating or the like using a thin film such as aluminum and silver. Thus, it is possible to reflect the laser light incident on the reflection surface 51 at high efficiency. Further, the surface of the reflection surface 51 may be appropriately subjected to protection coating or the like using a thin film such as a SiO₂ film and a polymerization film. Besides this, the material or the like of the high-reflection coating, protection coating, or the like is not limited.

The inner light guide 13 is a rod integrator that is arranged in the condensing region 4 and uniformizes and emits the incident light. The inner light guide 13 has an incident end 18, a light guide unit 19, and an emission end 20. In the present embodiment, a square-column-shaped rod integrator of which the end surface shape is a square is used as the inner light guide 13. Accordingly, the inner light guide 13 is a cuboid longitudinal member extending in one direction.

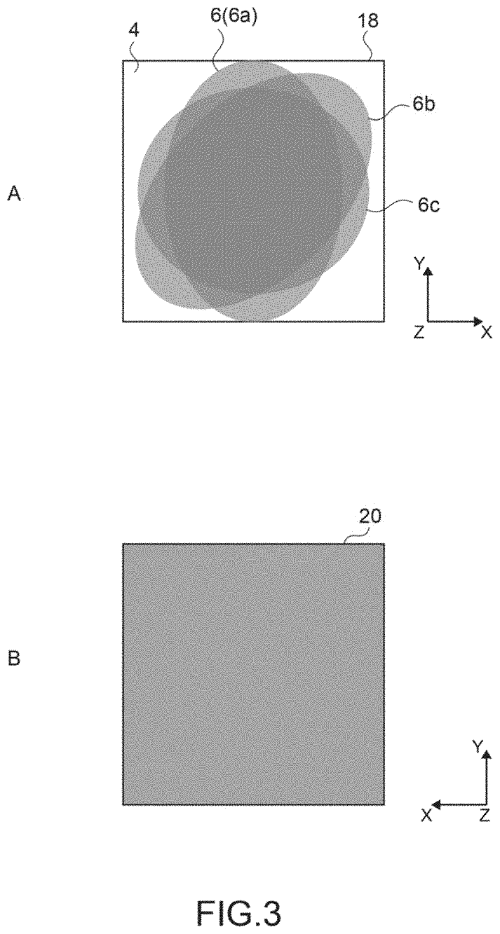

The incident end 18 is a square end surface provided at one end of the inner light guide 13 (see FIG. 3A). The light guide unit 19 guides the light incident from the incident end 18. Inside the light guide unit 19, the total reflection or the like of the light is repeatedly performed a plurality of times by four lateral surfaces to guide the light. The emission end 20 is a square end surface on a side opposite to the incident end 18 (see FIG. 3B). From the emission end 20, the light having passed through the light guide unit 19 is emitted. Hereinafter, an axis passing through the center of the incident end 18 and the center of the emission end 20 will be described as the central axis of the inner light guide 13. In the present embodiment, the central axis corresponds to a light guide axis that passes through the incident end and the emission end.

As shown in FIG. 1, the inner light guide 13 is arranged with the incident end 18 directed to the reflector 50 so that the central axis of the inner light guide 13 is coincident with the light axis 3. In other words, an axis obtained when the central axis of the reflector 50 (the reflection surface 51) and the central axis (light guide axis) of the inner light guide 13 described above are made coincident with each other is the light axis 3 of the light source unit 10.

The incident end 18 of the inner light guide 13 is an end surface on which the light condensed by the optical member 12 is incident, and is arranged in the condensing region 4. That is, a distance in the Z direction between the inner light guide 13 and the reflector 50 is set so that the focus P (the focus region 5) of the reflector 50 is coincident with the incident end 18. In other words, the condensing region 4 for the optical member 12 (the reflector 50) is set at the incident end 18. As a result, the laser light reflected by the first reflector 50 is condensed in the condensing region 4 on the incident end 18. As described above, the reflector 50 is arranged to reflect the light emitted from the plurality of laser diodes 15 and condense the reflected light on the incident end 18. Accordingly, in the present embodiment, the respective laser light is condensed on the incident end 18 of the inner light guide 13 only by the reflector 50. Thus, it is possible to reduce the number of parts for condensing the laser light and achieve a reduction in apparatus size or a reduction in apparatus cost.

Note that the incident end 18 and the focus P are not necessarily arranged to be coincident with each other. For example, when condensing accuracy is sufficiently high and a condensing spot is sufficiently small or when the area of the incident end 18 is sufficiently large, it is possible to properly condense the respective laser light on the incident end 18 even when the incident end 18 is slightly deviated from the focus P. As described above, the incident end 18 may be arranged near the focus P of the first reflector 50 as far as the exhibition of desired condensing efficiency or the like is, for example, made possible. That is, the coincidence between the incident end 18 and the focus P includes a case in which the incident end 18 and the focus P are made substantially coincident with each other.

The inner light guide 13 guides the light incident from the incident end 18 to the emission end 20. For example, the laser light condensed on the incident end 18 is incident on the light guide unit 19 from the incident end 18, guided toward the emission end 20 while being totally repeatedly reflected inside the light guide unit 19, and emitted from the emission end 20. Since the laser light is totally repeatedly reflected by the light guide unit 19, it is possible for the inner light guide 13 to emit uniform light. As described above, the inner light guide 13 uniformizes the condensed laser light incident on the incident end 18 and emits the uniformized laser light from the emission end 20. The operation of the inner light guide 13 will be described in detail later using FIGS. 3A and 3B or the like. In the present embodiment, the inner light guide 13 corresponds to a first light guide body.

The inner light guide 13 includes, for example, a quartz rod, a glass rod, or the like. Further, the areas of the respective end surfaces (the incident end 18 and the emission end 20) are appropriately set according to, for example, the condensing accuracy of the reflector 50, the area of the end surface of the outer light guide 31 that will be described later, or the like. Further, the length of the light guide unit 19 is appropriately set according to the number of total reflection times (uniformizing accuracy), or the like.

Besides this, a specific configuration such as the material and the shape of the inner light guide 13 is not limited. For example, the section of the inner light guide 13 is not limited to a square section, but a rod integrator having an arbitrary polygonal section may be used. Further, a tapered rod integrator or the like may be used. Alternatively, the lateral surfaces in the longitudinal direction may be subjected to coating or the like to prevent cracks or the like.

The relay optical system 30 is an optical system that connects the light emitted from the inner light guide 13 of the light source unit 10 to the outer light guide 31 on the subsequent stage. Specifically, the light emitted from the emission end 20 of the inner light guide 13 is connected to an incident end 33 of the outer light guide 31. As the relay optical system 30, an optical system that condenses again the light emitted from the inner light guide 13 is, for example, used.

A specific configuration of the relay optical system 30 is not limited, and the relay optical system 30 may perform, for example, arbitrary optical processing other than the connection of the light to the outer light guide 31 described above. The relay optical system 30 may have, for example, a diffusion element for unifying the diffusion angle of the light, a collimate optical system for parallelizing the light, a polarization control element for controlling a polarization direction, or the like. Further, the relay optical system 30 may have a multiplexing optical system for multiplexing the light emitted from the inner light guide 13 and light generated by other light sources together. In this case, the multiplexed light is condensed on the outer light guide 31. Besides this, the relay optical system 30 may have arbitrary optical elements and optical systems.

The outer light guide 31 guides the light to the observation target 2. As the outer light guide 31, a fiber bundle in which a plurality of optical fibers is bundled together is, for example, used. The fiber bundle is configured to be bendable and arranged inside the housing of an observation apparatus such as an endoscope (such as a soft endoscope and a hard endoscope) and a microscope for surgical operation. Of course, a light guide or the like other than the fiber bundle may be appropriately used according to the type of the observation apparatus.

The outer light guide 31 has the incident end 33 and an emission end 34. The incident end 33 and the emission end 34 include, for example, the sections of a plurality of optical fibers. The outer light guide 31 is arranged to make the emission end 34 placed on the side (for example, the tip side of an endoscope) directed to the observation target 2. On the incident end 33 of the outer light guide 31, the light having passed through the relay optical system 30 is condensed. The light incident on the incident end 33 is emitted from the emission end 34 through the respective optical fibers.

In the present embodiment, the area of the emission end 20 of the inner light guide 13 is configured to be smaller than that of the incident end of the outer light guide 31. That is, the respective light guides are configured so that the sectional size of the waveguide of the outer light guide 31 is larger than that of the waveguide of the inner light guide 13.

Thus, it is possible to sufficiently prevent the leakage of the light when the light is connected from the inner light guide 13 to the outer light guide 31, and is possible to remarkably improve the coupling between the respective light guides. As a result, it is possible to avoid a situation such as a reduction in the brightness of the irradiation light 1 irradiated on the observation target 2.

The illumination optical system 32 is an optical system that irradiates the observation target 2 with the light. The illumination optical system 32 includes an optical element such as a lens and an aperture and is provided at, for example, the tip or the like of an endoscope. In FIG. 1, the illumination optical system is schematically shown by a convex lens. The light emitted from the emission end 34 of the outer light guide 31 passes through the illumination optical system 32 and is irradiated onto the observation target 2 as the irradiation light 1. A specific configuration of the illumination optical system 32 is not limited.

For example, in order to make it possible to properly observe the observation target 2, an arbitrary optical system that enlarges or contracts the emitted light to be irradiated may be used.

The imaging element 40 shoots an operating field that is the observation target 2 of the medical observation system 100. For example, the light emitted from the inner light guide 13 (the light source unit 10) is irradiated onto the observation target 2 via the outer light guide 31, the illumination optical system 32, or the like. The imaging element 40 shoots the operating field of the observation target 2 using the light as illumination light. As described above, the imaging element 40 irradiates the operating field with the light guided to the emission end 20 and captures an image of light reflected from a subject.

As the imaging element 40, a digital camera or the like using an image sensor such as a CCD (Charge Coupled Device) sensor and a CMOS (Complementary Metal-Oxide Semiconductor) sensor is, for example, used. Alternatively, a camera or the like capable of capturing an image of light outside a visible range such as an infrared camera and an ultraviolet camera may be used. Note that a method or the like for guiding the light to the imaging element 40 is not limited. For example, reflection light or the like for shooting may be guided by an optical system common to an irradiation system, or a configuration in which an image is directly shot with the imaging element 40 arranged near an operating field may be employed. Besides this, the imaging element 40 may be appropriately configured according to the type or the like of the system.

FIGS. 3A and 3B are schematic views each showing an example of the brightness distribution of an end surface of the inner light guide 13. In FIGS. 3A and 3B, the brightness distribution of the incident end 18 and the brightness distribution of the emission end 20 of the inner light guide 13 are schematically shown by a gray scale, respectively.

In the present embodiment, the plurality of laser light reflected by the reflector 50 (the reflection surface 51) is condensed on the incident end 18 as described above. In FIG. 3A, spots 6 (spots 6a, 6b, and 6c) of three laser light condensed on the incident end 18 are schematically shown. Actually, the plurality of spots 6 of the laser light emitted from the plurality of laser diodes 15 is formed on the incident end 18. In the present embodiment, the reflector 50 is arranged so that the respective spots 6 of the laser light emitted from the plurality of laser diodes 15 overlap each other on the end surface of the incident end 18.

As shown in FIG. 3A, a state in which the spots 6a to 6c overlap each other is realized on the incident end 18. In other words, the laser light emitted from the different laser diodes 15 is multiplexed together at a portion at which the respective spots 6a to 6c overlap each other. For example, it is assumed that the spots 6a to 6c are the spots 6 of the laser diodes 15R, 15G, and 15B that emit red light, green light, and blue light, respectively. In this case, the red light, the green light, and the blue light are multiplexed together to generate white light at the portion at which the respective spots 6a to 6c overlap each other.

As described above, the use of the reflector 50 shown in FIG. 1 makes it possible to directly multiplex together the laser light emitted from the plurality of laser diodes 15 on the incident end 18 (the condensing region 4). That is, since the light outputs of the plurality of laser diodes 15 are multiplexed together at the same time by the one reflector, it is possible to generate white light or the like with fewer parts. That is, in the present embodiment, it is possible to generate the white light, in which the respective laser light has been multiplexed together, at a time using the single reflector 50 rather than successively multiplexing the respective laser light together. Thus, it is possible to reduce an apparatus size and achieve a reduction in the number of parts and an apparatus cost.

Further, the respective laser light is condensed by reflection. The condensing of the light by reflection makes it possible to easily change the traveling direction of the laser light to an arbitrary direction. Therefore, it is possible to remarkably shorten a distance for the condensing and sufficiently shorten, for example, the distance between the inner light guide 13 and the reflector 50. As a result, it is possible to sufficiently reduce the size in the longitudinal (Z direction) of the light source unit 10 and sufficiently reduce an apparatus size.

The respective laser light reflected by the reflector 50 is incident on the incident end 18 from different directions. For example, light axes that are the traveling directions of the respective laser light are different in incident angle, direction, or the like from each other with respect to the incident end 18. As described above, the optical member 12 is arranged to reflect the laser light emitted from the plurality of laser diodes 15 and make the respective light axes of the reflected laser light incident on the incident end from different directions.

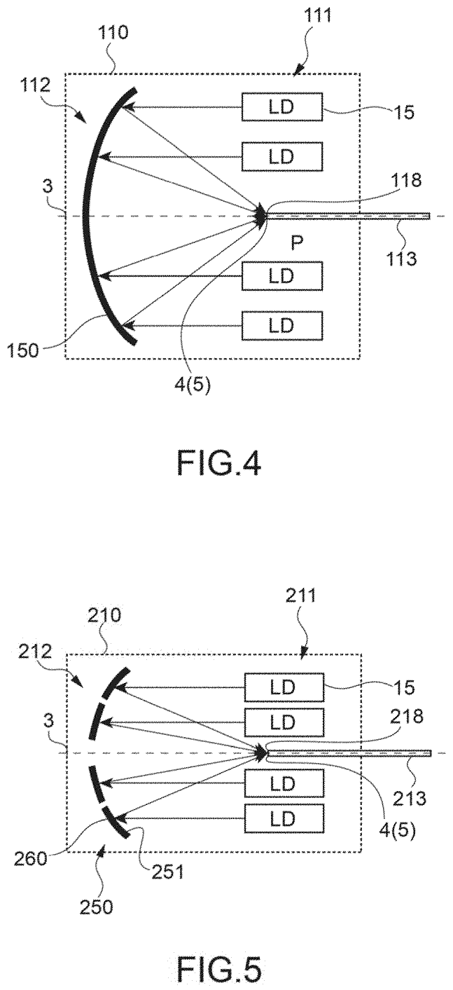

As described above, it is possible to make the respective laser light incident from different directions. Therefore, for example, when the number of the laser diodes 15 increases (see FIG. 4 or the like) or when the interval between the laser diodes 15 is narrow (see FIG. 5 or the like), it is possible to properly condense the light on the incident end 18 without disturbing the light paths of the respective laser light. Thus, it is possible to configure a high-brightness light source unit or reduce an apparatus size.

Further, the laser diodes 15 are smaller in the sizes of light-emitting points or the radiation angle of the light than other light sources such as lamp light sources and LED light sources. Therefore, since the sizes of the spots 6 at which the respective laser light is condensed are small, it is possible to put the light of respective wavelength ranges together in a smaller region. Therefore, it is possible to efficiently guide the light, for example, when the incident end 18 has a small area.

Note that the spots 6a to 6c of the respective laser light condensed on the incident end 18 do not necessarily completely overlap each other. In, for example, FIG. 3A, the spot 6a has an elliptical shape long in the vertical direction on the incident end 18, the spot 6b has an elliptical shape long in the horizontal direction (X direction) on the incident end 18, and the spot 6c has an elliptical shape long in an oblique direction from the lower left to the upper right in the figure on the incident end 18.

As a result, a region in which only the light of the spot 6a is incident, a region in which the light of the spots 6a and 6b is incident, or the like is, for example, formed on the incident end 18. These regions are different in brightness or color from a region in which white light is generated. As described above, it is presumed that brightness unevenness, color unevenness, or the like is caused in a state in which the respective laser has been multiplexed together at the incident end 18.

Generally, the radiation angles of the laser light (the spread angles of the beams) or the sizes of light-emitting points (stripe widths) are different depending on the types of the laser diodes 15. Therefore, laser light having different beam shapes are emitted from different types of the laser diodes 15. As a result, for example, the spots 6 of the laser light emitted from the different types of the laser diodes 15 have different shapes. Note that the beam shapes of the laser light could be different from each other due to individual differences, operation environments, or the like even among the same type of elements.

Further, for example, even laser light having a circular beam shape forms the spot 6 having an elliptical shape when being incident on the incident end 18 obliquely. The larger an incident angle with respect to the incident end 18, the larger the deformation at the incident becomes. Note that the incident angle is the angle between the incident direction (light path) of the laser light and the normal direction of the incident end 18 (the direction parallel to the light axis 3). As described above, it is presumed that the shape of the spot 6 could also be changed due to a difference in the incident angle with respect to the incident end 18 of the laser light.

The light condensed on the incident end 18 (the condensing region 4) is incident on the inner light guide 13. For example, it is assumed that the laser light forming the spot 6a is incident on the incident end 18 at a certain incident angle. The laser light is guided toward the emission end 20 while being totally repeatedly reflected a plurality of times by the four lateral surfaces of the inner light guide 13. When the laser light is guided by the inner light guide 13, that is, by the light guide unit 19 (waveguide), a plurality of spot images is generated at the emission end by multiple reflection and the laser light is uniformized according to a superimposing effect.

Similarly, the laser light forming other spots 6 is also uniformized while being guided toward the emission end 20.

As a result, the light having uniform brightness distribution is emitted from the emission end 20 as shown in FIG. 3B. That is, the inner light guide 13 uniformizes the brightness distribution at the emission end 20 of the light emitted from the emission end 20. As described above, the inner light guide 13 produces, even when the shapes of the spots 6 at the incident end 18 are different from each other, the effect of uniformizing the laser light while guiding the same and changing the spots 6 of the respective laser light at the emission end 20 into shapes matching the shape of the end surface of the inner light guide 13.

Thus, for example, when the light of the respective colors of RGB is incident on the incident end 18, it is possible to emit high-quality white light having no color unevenness. In other words, the inner light guide 13 multiplexes the light of respective wavelength ranges together at the emission end 20. As a result, it is possible to perform, for example, the illumination of the observation target 2 at high accuracy and shoot a high-quality observation image.

Further, the uniformity of the brightness distribution at the emission end 20 is improved as the number of times of total reflection occurring inside the inner light guide 13 (the waveguide unit 19) increases. In the present embodiment, the use of the reflector 50 makes it possible to increase the incident angle with respect to the incident end 18. As a result, the number of reflection times inside the inner light guide 13 increases, which makes it possible to increase the uniformity of the brightness distribution. Alternatively, since the incident angle of the laser light is large, it is possible to secure sufficient uniformity even when the inner light guide 13 that is relatively short is, for example, used. Accordingly, it is possible to reduce the length of the inner light guide 13 within an allowable range and reduce an apparatus size.

Note that the emission direction (emission angle) of the light emitted from the emission end 20 depends on the incident angle of the respective laser light when being incident on the incident end 18. Here, the emission angle is the angle between the emission direction of the light and the normal direction of the emission end 20. For example, a light component (laser light) incident at a small incident angle turns into light that is to be emitted at a small emission angle. Conversely, a light component incident at a large incident angle is to be emitted at a large emission angle.

For example, when red laser light is deviated to be incident at a small angle, there is a possibility that light having a small emission angle and emitted in a direction close to the light axis 3 among the light emitted from the emission end 20 is deviated to red. As described above, when the incident direction of the laser light of a certain color (wavelength range) is deviated, an emission direction could be deviated according to the deviation.

In the present embodiment, the respective types of the laser diodes 15 are arranged so that the characteristics of the light paths of the laser light of respective wavelength ranges become uniform as described with reference to FIGS. 2A and 2B.

In, for example, FIG. 2A, each of the five types of the laser diodes 15 (laser diodes 15R, 15G, 15B, 15IR, and 15UV) is arranged at an arrangement position on the same circle. As a result, the incident angles of the laser light of the respective wavelength ranges with respect to the incident end 18 become substantially equal to each other between the light paths of all the laser light. As a result, it is possible to sufficiently reduce the deviation or the like of an angle for each wavelength range of the light emitted from the emission end 20.

Further, in FIG. 2B, at least one of the five types of the laser diodes 15 is arranged at an arrangement position outside a lattice-shaped arrangement position. Thus, as for at least one of the laser light of the respective wavelength ranges, it is possible to make an incident angle with respect to the incident end 18 fall within a constant angle range. As a result, the deviation of the incident angle is alleviated, which makes it possible to reduce the deviation of an angle for each wavelength range of the light emitted from the emission end 20.

As described above, the plurality of laser diodes 15 is arranged so that the incident angle of the light emitted from at least one of the same type of the laser diodes 15 with respect to the incident end 18 falls within a constant angle range in the present embodiment.

The constant angle range is, for example, an angle range at which the deviation of an emission direction at the emission end falls within an allowable range. For example, in order to make it possible to properly perform the irradiation of white light, a constant angle range is appropriately set. In the present embodiment, the constant angle range corresponds to a prescribed range.

Note that, for example, the provision of a diffusion element or the like in the relay optical system 30 makes it possible to reduce the deviation or the like of the emission direction of the light emitted from the inner light guide 13 (the emission end 20). Thus, it is possible to supply high-quality white light having no color unevenness or the like.

FIGS. 4 to 8 are schematic views each showing another configuration example of the light source unit. Light emitted from respective light source units 110 to 510 shown in FIGS. 4 to 8 is incident on the outer light guide 31 via the relay optical system 30 and irradiated onto the observation target 2 from the illumination optical system 32 as the irradiation light 1. Note that in FIGS. 4 to 8, the diagrammatic representation of a radiation unit on which laser diodes 15 are arranged is omitted.

As shown in FIG. 4, the light source unit 110 has a light source 111 including a plurality of laser diodes 15, an optical member 112 including a reflector 150, and an inner light guide 113. The optical member 112 (the reflector 150) and the inner light guide 113 are configured like, for example, the optical member 12 and the inner light guide 13 of the light source unit 10 shown in FIG. 1, respectively.

In the light source unit 110, four laser diodes 15 are arranged in a section including a light axis 3. In this configuration example, the number of the laser diodes 15 is increased as compared with, for example, the light source unit 10 shown in FIG. 1.

As shown in FIG. 4, the plurality of laser diodes 15 is arranged toward the reflector 150 around the light axis 3 in the light source unit 110. In other words, the plurality of laser diodes 15 is arranged around the light guide axis of the inner light guide 113. Further, the plurality of laser diodes 15 emits laser light parallel to the light axis 3 toward the rear side of the light source unit 110. Accordingly, the plurality of laser light parallel to the light axis 3 is incident on the reflector 150. The laser light reflected by the reflector 150 is condensed toward a condensing region 4, that is, toward an incident end 118 of the inner light guide.

As described above, it is possible to easily condense the respective laser light toward the incident end 118 even when the number of the laser diodes 15 around the light axis 3 is increased. Thus, it is possible to easily realize, for example, the high brightness of white light with the additional installation of laser diodes 15R to 15B, multifunction with the addition of laser diodes 15IR and 15UV that emit infrared light and ultraviolet light, respectively, or the like.

As shown in FIG. 5, a light source unit 210 has a light source 211, an optical member 212, and an inner light guide 213. The inner light guide 213 is configured like, for example, the inner light guide 13 shown in FIG. 1. The light source 211 has a plurality of laser diodes 15. In the light source 211, the respective laser diodes 15 are configured to be arranged at a shorter and denser distance as compared with, for example, the light source 111 of the light source unit 110 shown in FIG. 4.

The optical member 212 has a reflector 250. The reflector 250 includes a plurality of divided mirrors 260. In the light source unit 210, a free-form surface mirror is used as the reflector 250. That is, the free-form surface mirror including the plurality of divided mirrors 260 is used as the reflector 250.

Here, the free-form surface mirror is a mirror including a free-form surface as its reflection surface. The free-form surface mirror (the reflector 250) is designed to reflect laser light incident parallel to a light axis 3 and condense the light in a focus region 5 on the light axis 3. The designing of such a free-form surface is made possible by, for example, a light path simulation or the like.

The plurality of divided mirrors 260 has respective reflection surfaces 251 and is arranged facing the plurality of laser diodes 15 with the reflection surfaces 251 directed to the emission side of the laser light. In FIG. 5, the sections of the four divided mirrors 260 arranged facing the four laser diodes 15, respectively, are schematically shown. On the reflection surfaces 251 of the respective divided mirrors 260, the laser light emitted from the corresponding laser diodes 15 is incident.

Note that each one of the divided mirrors 260 is not necessarily arranged with respect to each one of the laser diodes 15. For example, a configuration in which the laser light emitted from two or more of the laser diodes 15 is reflected by one divided mirror 260 may be, for example, employed.

As the reflection surfaces 251 of the divided mirrors 260, curved surfaces, planes, or the like capable of reflecting the incident laser light toward the focus region 5 are, for example, used. By these reflection surfaces 251, the free-form surface that is discontinuous is constituted. As described above, the free-form surface mirror is constituted by the discontinuous and independent divided mirrors 260 in the light source unit 210.

The laser light emitted parallel to the light axis 3 from the plurality of laser diodes 15 is reflected by the respective divided mirrors 260 and condensed in the focus region 5 (condensing region 4) of the free-form surface. The condensed light is incident on an incident end 218 of an inner light guide 213 that is arranged in the focus region 5.

In the light source unit 210, for example, the appropriate adjustment of the angles, positions, or the like of the respective divided mirrors 260 makes it possible to adjust the positions, shapes, or the like of spots 6 of the laser light condensed on the incident end 218. Thus, it is possible to perform, for example, a reduction in a range in which the respective laser light is to be condensed and the efficient guidance of the laser light to the inner light guide 213 that is thin.

Further, the use of the divided mirrors 260 makes it possible to configure, for example, the reflector 250 (free-form surface mirror) to be small. Thus, for example, since it is possible to make the arrangement distances between the adjacent laser diodes 15 smaller, a further reduction in the size of the light source unit 210 is allowed.

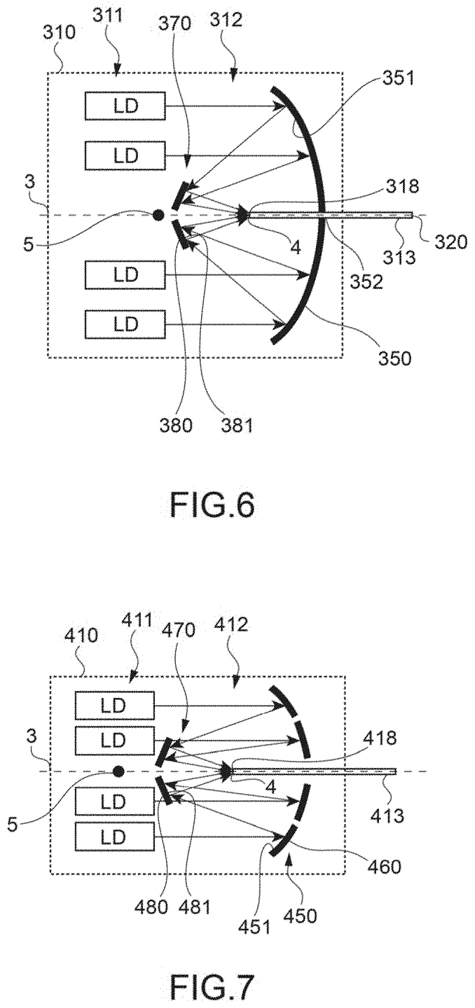

As shown in FIG. 6, a light source unit 310 has a light source 311, an optical member 312, and an inner light guide 313. The inner light guide 313 is configured like, for example, the inner light guide 13 shown in FIG. 1. Hereinafter, a description will be made assuming that sides on which an incident end 318 and an emission end 320 of the inner light guide 313 are provided are the rear side and the front side of the light source unit 310, respectively.

The light source 311 has a plurality of laser diodes 15. The plurality of laser diodes 15 emits laser light parallel to the light axis 3 toward the front side of the light source unit 310.

The optical member 312 has a first reflector 350 and a second reflector 370. The first reflector 350 is rotationally-symmetric parabolic mirror and has a first reflection surface 351. The first reflector 350 is configured like, for example, the reflector 50 described with reference to FIG. 1. The first reflector 350 is arranged so that its central axis is coincident with a light axis 3 with the first reflection surface 351 directed to the emission side of the laser diodes 15 (directed to the rear side of the light source unit 310).

Note that the first reflector 350 has an opening part 352 at its central area of, that is, at its area crossing the light axis 3. The opening part 352 is, for example, a square-shaped through-hole, and the inner light guide 313 is inserted into the opening part 352. The inner light guide 313 inserted into the opening part 352 is arranged to make its central axis coincident with the light axis 3. Note that the incident end 318 of the inner light guide 313 is arranged at a position closer to the front side than a focus region 5 of the first reflector 350.

The second reflector 370 is arranged facing the first reflector 350. In an example shown in FIG. 6, the second reflector 370 includes a plurality of divided mirrors 380. The plurality of divided mirrors 380 has respective reflection surfaces 381. Hereinafter, the divided mirrors 380 and the reflection surfaces 381 constituting the second reflector 370 will be described as second divided mirrors 380 and second reflection surfaces 381, respectively. In the present embodiment, the second reflector 370 corresponds to a second reflection unit.

The second divided mirrors 380 are arranged so that the laser light reflected by the first reflector 350 is incident on the second reflection surfaces 381. That is, the second divided mirrors 380 (the second reflector 370) are arranged on the light paths of the laser light reflected by the first reflector 350 and condensed toward the focus region 5.

Further, the second divided mirrors 380 are configured to reflect the incident laser light toward the incident end 318 (the condensing region 4) of the inner light guide 313. Accordingly, the laser light reflected by the first reflector 350 (parabolic mirror or the like) is condensed on the incident end 318 of the inner light guide 313 after being reflected by the second divided mirrors 380.

As the second divided mirrors 380, plane mirrors are, for example, used. In this case, the second reflection surfaces 381 are plane-shaped reflection mirrors. The use of the plane mirrors makes it possible to directly fold back, for example, the light paths of the laser light condensed toward the focus region 5. Further, as the second divided mirrors 380, parabolic mirrors, free-form surface mirrors, or the like may be used. Thus, it is possible to perform, for example, condensing of the incident laser light toward the condensing region 4 again and exhibit high condensing efficiency.

Note that the second reflector 370 may include an undivided mirror. That is, as the second reflector 3701, a single plane mirror, a parabolic mirror, a free-form surface mirror, or the like may be used. In this case as well, the appropriate configuration of the second reflector 370 makes it possible to properly condense the laser light on the incident end 318 of the inner light guide 313. Besides this, a specific configuration of the second reflector 370 is not limited.

As described above, the laser light directed from the first reflector 350 to the focus region 5 is reflected toward the condensing region 4 by the second reflector 370 in the light source unit 310. The folding back of the light paths of the laser light with the second reflector 370 makes it possible to perform, for example, sufficiently shortening of a distance for condensing. As a result, it is possible to sufficiently reduce an apparatus size.

Further, for example, the appropriate adjustment of the position, angle, or the like of the second reflector makes it possible to adjust a condensing position with respect to the condensing region 4 (the incident end 318 of the inner light guide 313). As a result, it is possible to increase the amount of the laser light incident on the inner light guide 313 and increase optics use efficiency. Further, since the slight adjustment of the condensing position is made possible, it is also possible to properly introduce the laser light into the inner light guide 313 that is thin.

As shown in FIG. 7, a light source unit 410 has a light source 411, an optical member 412, and an inner light guide 413. The inner light guide 413 is configured like, for example, the inner light guide 13 shown in FIG. 1. The light source 411 has a plurality of laser diodes 15. The plurality of laser diodes 15 emits laser light parallel to a light axis 3 toward the front side of the light source unit 410.

The optical member 412 has a first reflector 450 and a second reflector 470. The first reflector 450 is a free-form surface mirror including a plurality of divided mirrors 460 (first divided mirrors 460) and has first reflection surfaces 451. The first reflector 450 is configured like, for example, the reflector 250 described with reference to FIG. 5. The first reflector 450 is arranged so that its central axis is coincident with a light axis 3 with the first reflection surfaces 451 directed to the emission side of the laser diodes 15 (directed to the rear side of the light source unit 410).

Further, the inner light guide 413 is arranged at the central area of the first reflector 450 along the light axis 3. Note that an incident end 418 of the inner light guide 413 is arranged at a position closer to the front side than a focus region 5 of the first reflector 450.

The second reflector 470 includes a plurality of second divided mirrors 480. The second reflector 470 is arranged on the light paths of the laser light condensed toward the focus region 5 with second surfaces 481 of the second divided mirrors 480 directed to the first reflection surfaces 451 (see FIG. 5). Further, the second reflector 470 is configured to reflect the incident laser light toward the incident end 418 (the condensing region 4) of the inner light guide 413.

Even in a configuration in which the free-form surface mirror is used as the first reflector 450 as described above, the provision of the second reflector 470 makes it possible to fold back the laser light to be condensed toward the incident end 418 of the inner light guide 413. The use of the free-form surface mirror makes it possible to reduce the sizes in the horizontal direction (X direction) and the vertical direction (Y direction) of the light source unit 410. Further, the use of the second reflector 470 makes it possible to reduce the size in the longitudinal direction (Z direction) of the light source unit 410. Thus, it is possible to remarkably reduce an apparatus size.

Further, in an example shown in FIG. 7, the first reflector 450 includes the first divided mirrors 460. Thus, for example, the appropriate adjustment of the first divided mirrors 460 and the second divided mirrors makes it possible to control the condensing positions or the like of the laser light in detail on the incident end 418. As a result, it is possible to highly efficiently introduce the laser light emitted from the respective laser diodes 15 into the inner light guide 413 and remarkably improve optics use efficiency.

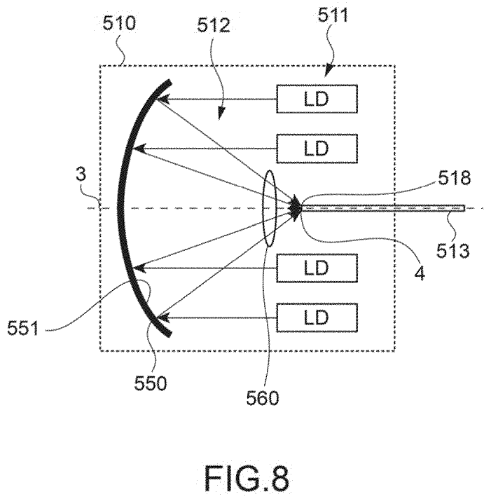

As shown in FIG. 8, a light source unit 510 has a light source 511, an optical member 512, and an inner light guide 513. The light source 511 and the inner light guide 513 are configured like, for example, the light source 111 shown in FIG. 4 and the inner light guide 13 shown in FIG. 1, respectively.

The optical member 512 has a reflector 550 and a lens unit 560. The reflector 550 is a rotationally-symmetric parabolic mirror and has a reflection surface 551. The reflector 550 is configured like, for example, the reflector 50 described with reference to FIG. 1 or the like. That is, the reflector 550 is arranged so that its central axis is coincident with a light axis 3 with the reflection surface 551 directed to the emission side of laser diodes 15 (directed to the front side of the light source unit 510).

For example, the laser light emitted parallel to the light axis 3 from the plurality of laser diodes 15 toward the rear side of the light source unit 510 is reflected by the reflection surface 551 of the reflector 550 toward an incident end 518 (a condensing region 4) of the inner light guide 513 positioned on the front side of the reflection surface 551. More specifically, the respective laser light is condensed toward the focus region (not shown) of the reflector 550 that is set near the incident end 518.

The lens unit 560 is arranged on the light paths of the laser light reflected toward the condensing region 4. That is, the lens unit 560 is arranged on the light axis 3 between the reflector 550 and the incident end 518 so that the respective laser light is incident on the lens unit 560.

The lens unit 560 condenses the laser light reflected from the reflector 550 toward the condensing region 4 on the condensing region 4. As shown in, for example, FIG. 8, the plurality of laser light reflected by the reflector 550 is radially incident on the lens unit 560. The lens unit 560 is appropriately configured so that such laser light is condensed on the incident end 518 of the inner light guide 513 that is the condensing region 4.

In FIG. 8, a single lens is schematically shown as the lens unit 560. Besides this, the lens unit 560 may include, for example, a plurality of optical elements containing a lens.

The lens unit 560 typically includes a condensing lens or the like. In this case, a region including the focus of the lens unit 560 serves as the condensing region 4. That is, the incident end 518 of the inner light guide 513 is arranged to be coincident with the focus position of the lens unit 560. Thus, it is possible to further reduce, for example, the sizes or the like of spots 6 of the laser light condensed on the incident end 518. Alternatively, it is possible to control the incident angles or the like of the respective laser light when being incident on the incident end 518.

Note that the light source unit 510 shown in FIG. 8 has a configuration in which the lens unit 560 is added to, for example, the configuration of the light source unit 110 described with reference to FIG.

4. For example, in the light source units 10, 210, 310, 410, or the like described with reference to FIG. 1, FIGS. 5 to 7, or the like, the lens unit 560 may be, for example, provided near the incident ends of the respective inner light guides. In this case, the positions or the like of the incident ends are appropriately adjusted according to the characteristics (the focus position) of the lens unit 560. Thus, it is possible to easily improve the condensing efficiency or the like of the laser light using the lens unit 560.

As described with reference to FIG. 1 and FIGS. 4 to 8, an optical member including one or more optical elements such as a reflector, divided mirrors, and a lens unit is constituted in the present embodiment. Further, the optical member is arranged so that respective laser light emitted from a plurality of laser diodes passes through the same number of optical elements. That is, all the laser light emitted from the respective laser diodes pass through the same number (the same type) of optical elements until the laser light is condensed after being emitted. Accordingly, the respective laser light passes through light paths having the same characteristics and is multiplexed together. As a result, it is possible to easily generate high-quality white light or the like.

Further, it is possible to multiplex all laser light together at a time with fewer parts as compared with, for example, a configuration in which respective laser light is successively multiplexed together to form white light. Thus, it is possible to reduce an apparatus cost or an apparatus size and realize a light source unit or the like that facilitates maintenance or the like.

As described above, the laser light emitted from the plurality of laser diodes 15 is reflected by the optical member and condensed in the condensing region 4 in the medical observation system 100 according to the present embodiment. The condensed laser light is incident on the incident end of the inner light guide arranged in the condensing region 4 and emitted from the emission end after being uniformized. As described above, the reflection of the laser light makes it possible to shorten a distance for condensing. Further, the laser light condensed by the inner light guide is uniformized as it is. Thus, it is possible to reduce an apparatus size and realize excellent observation.

It is presumed that a lamp light source (a xenon lamp or a halogen lamp), a white LED, or the like is used as the light source of an observation apparatus such as an endoscope and a microscope. It has been known that such a light source has a wide radiation angle due to its large light-emitting point. This represents that an etendue (the product of the area of a light flux and the spread angle (solid angle) of light) is large on the side of a light source. For example, when the etendue is large on the side of the light source, there is a possibility that the ratio of light capable of being not captured increases in an optical system that captures the light of the light source. Therefore, it could be difficult to efficiently condense light on a light guide or the like having a prescribed size.

Further, it is presumed that a lens condensing system is used as a method for condensing light from a light source on a light guide or the like. When the lens condensing system is used, it is difficult to suddenly change the light path of light and has to keep a distance to condense the light. Further, when the number of light sources increases, a situation such as an increase in the number of condensing lenses and an increase in the size of a condensing lens itself is likely to occur, which may result in an increase in the entire size.

In the present embodiment, the laser light emitted from the plurality of laser diodes 15 is condensed on the incident end (in the condensing region 4) of the inner light guide by being reflected by the parabolic mirror (reflector) or the like of the optical member. Thus, it is possible to arbitrarily change the light paths or the like of the laser light and condense the respective laser light at a short distance. As a result, it is possible to sufficiently reduce the size of the light source unit.

Further, as described using FIG. 3A or the like, the laser diodes 15 are light sources having a small light-emitting point and having a narrow radiation angle. In other words, the laser diodes 15 are light sources having a small etendue. Therefore, the use of the laser diodes 15 makes it possible to condense the laser light in a state in which the spread or the like of beam (spots) is sufficiently small and sufficiently increase the efficiency of condensing the light with respect to the inner light guide.

In the present embodiment, the brightness distribution of the laser light of respective wavelength ranges is uniformized by the inner light guide. Thus, it is possible to sufficiently reduce the color unevenness or the like of white light caused by a difference in beam shapes corresponding to the types of the laser diodes 15. As a result, it is possible to properly illuminate the observation target 2 and realize excellent observation.

For example, observation by endoscopes has become rapidly pervasive with the development of techniques in medical fields, and has become important observation means in many medical examination fields. It is desirable that such endoscopic observation apparatuses have low invasiveness to patients regardless of whether they have a soft mirror or a hard mirror. For example, thinning or miniaturization of scope portions that come in direct contact with patients has been advanced.

In the present embodiment, laser light is reflected and condensed. Therefore, the laser light is introduced also into a sufficiently-thin inner light guide at high condensing efficiency. Further, light having uniform brightness distribution is generated by the inner light guide. Thus, it is possible to emit the light having high brightness and uniform brightness distribution from the sufficiently-thin inner light guide.

Further, the size of the emission end of the inner light guide is set to be smaller than that of the incident end 33 of the outer light guide 31. Thus, it is possible to achieve excellent coupling even when the light is introduced into the outer light guide 31 (such as a fiber bundle) that is thin. As a result, it is also possible to efficiently guide the light to a light guide used in a thin endoscope or the like having low invasiveness. Thus, it is possible to realize sufficiently excellent observation such as shooting a high-quality observation image even under low invasiveness.

### Other Embodiments

The present technology is not limited to the embodiment described above but is capable of realizing various other embodiments.

In the above embodiment, a rod integrator such as a quartz rod and a glass rod is used as the inner light guide. Besides this, an arbitrary optical element that uniformizes and emits incident light may be used as the inner light guide.

An optical fiber may be, for example, used as the inner light guide. Thus, the inner light guide is, for example, configured to be bendable and capable of being easily connected to the subsequent optical system. Further, a hollow mirror or the like having a reflection surface on its square tube reflection surface may be used as the inner light guide. The use of the hollow mirror or the like makes it possible to achieve the weight reduction of the apparatus.

In the above embodiment, the laser light is emitted parallel to the light axis from the respective laser diodes. Besides this, the emission directions of the respective laser light may be arbitrarily set. For example, the plurality of laser light may be emitted to diverge and converge about the light axis. Even in such a configuration, the optical member (such as a reflector) appropriately including a free-form surface mirror or the like makes it possible to condense the laser light in a desirable condensing region. Such a configuration may be, for example, employed.

In FIGS. 2A and 2B, the five types of the laser diodes that emit red light, green light, blue light, infrared light, and ultraviolet light are used as the plurality of laser diodes. Besides this, light sources may include, for example, one type of laser diodes. Even in such a case, it is possible to efficiently condense the laser light emitted from the multiplicity of laser diodes on the inner light guide and easily generate single-color irradiation light or the like that is bright and has small brightness unevenness.

Further, a configuration in which laser diodes that emit red light, green light, and blue light are mounted to emit white light, a configuration in which laser diodes that emit infrared light and ultraviolet light are mounted to generate irradiation light for specific observation, or the like may be used. Besides this, a configuration in which various types of laser diodes are arbitrarily combined together according to the purpose of a medical light source unit may be employed.

Further, light-emitting elements other than laser diodes may be used. It is possible to use, for example, LED elements or the like instead of laser diodes. In this case, LED elements capable of emitting red light, green light, blue light, infrared light, ultraviolet light, or the like may be appropriately used. Alternatively, white LEDs or the like capable of emitting white light may be used. Even in a case in which the LED elements are used as described above, it is possible to efficiently condense light on the incident end of the inner light guide.

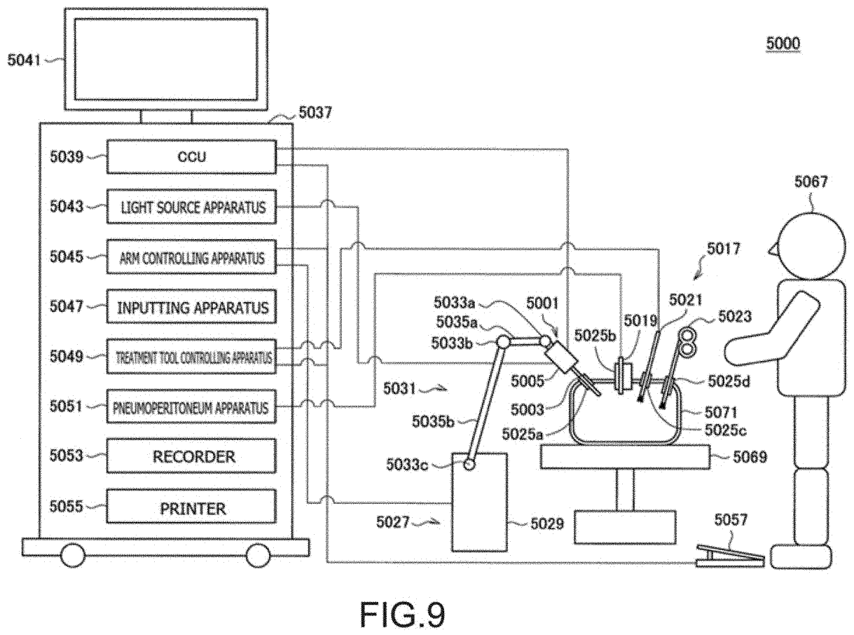

FIG. 9 is a view depicting an example of a schematic configuration of an endoscopic surgery system 5000 according to another embodiment. In FIG. 9, a state is illustrated in which a surgeon (medical doctor) 5067 is using the endoscopic surgery system 5000 to perform surgery for a patient 5071 on a patient bed 5069. As depicted, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body lumens of the patient 5071 through the trocars 5025a to 5025d.

In the example depicted, as the other surgical tools 5017, a pneumoperitoneum tube 5019, an energy treatment tool 5021 and forceps 5023 are inserted into body lumens of the patient 5071. Further, the energy treatment tool 5021 is a treatment tool for performing incision and peeling of a tissue, sealing of a blood vessel or the like by high frequency current or ultrasonic vibration. However, the surgical tools 5017 depicted are mere examples at all, and as the surgical tools 5017, various surgical tools which are generally used in endoscopic surgery such as, for example, a pair of tweezers or a retractor may be used.

An image of a surgical region in a body lumen of the patient 5071 imaged by the endoscope 5001 is displayed on a display apparatus 5041. The surgeon 5067 would use the energy treatment tool 5021 or the forceps 5023 while watching the image of the surgical region displayed on the display apparatus 5041 on the real time basis to perform such treatment as, for example, resection of an affected area. It is to be noted that, though not depicted, the pneumoperitoneum tube 5019, the energy treatment tool 5021 and the forceps 5023 are supported by the surgeon 5067, an assistant or the like during surgery.

The supporting arm apparatus 5027 includes an arm unit 5031 extending from a base unit 5029. In the example depicted, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

The endoscope 5001 includes the lens barrel 5003 which has a region of a predetermined length from a distal end thereof to be inserted into a body lumen of the patient 5071, and a camera head 5005 connected to a proximal end of the lens barrel 5003. In the example depicted, the endoscope 5001 is depicted which includes as a hard mirror having the lens barrel 5003 of the hard type. However, the endoscope 5001 may otherwise be configured as a soft mirror having the lens barrel 5003 of the soft type.

The CCU 5039 includes a central processing unit (CPU), a graphics processing unit (GPU) or the like and integrally controls operation of the endoscope 5001 and the display apparatus 5041. The display apparatus 5041 displays an image based on an image signal for which the image processes have been performed by the CCU 5039 under the control of the CCU 5039.

A light source apparatus 5043 includes the medical observation system 100 depicted in, for example, FIG. 1. In other words, the light source apparatus 5043 includes the light source unit 10, the relay optical system 30, the outer light guide 31, and the like. Further, a controller that individually controls the laser diodes 15 of the light source unit 10, or the like is provided as the light source apparatus 5043. It is to be noted that the illumination optical system 32 includes an objective lens provided to the distal end of the endoscope 5001, or the like.

Further, the light source apparatus 5043 may be provided at a place different from the cart 5037. For example, the light source unit 10 and the relay optical system 30 may be provided in the base unit 5029 of the supporting arm apparatus 5027. In this case, the outer light guide 31 of the soft type is inserted to the distal end portion of the endoscope 5001 through the inside and vicinity of the arm unit 5031. Further, for example, the light source apparatus 5043 may be provided in another casing and connected to the endoscope 5001 via the outer light guide 31.

The arm controlling apparatus 5045 includes a processor such as, for example, a CPU and operates in accordance with a predetermined program to control driving of the arm unit 5031 of the supporting arm apparatus 5027 in accordance with a predetermined controlling method. An inputting apparatus 5047 is an input interface for the endoscopic surgery system 5000. A user can perform inputting of various kinds of information or instruction inputting to the endoscopic surgery system 5000 through the inputting apparatus 5047. As the inputting apparatus 5047, for example, a mouse, a keyboard, a touch panel, a switch, a foot switch 5057 and/or a lever or the like may be applied.

A treatment tool controlling apparatus 5049 controls driving of the energy treatment tool 5021 for cautery or incision of a tissue, sealing of a blood vessel or the like. A pneumoperitoneum apparatus 5051 feeds gas into a body lumen of the patient 5071 through the pneumoperitoneum tube 5019 to inflate the body lumen in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

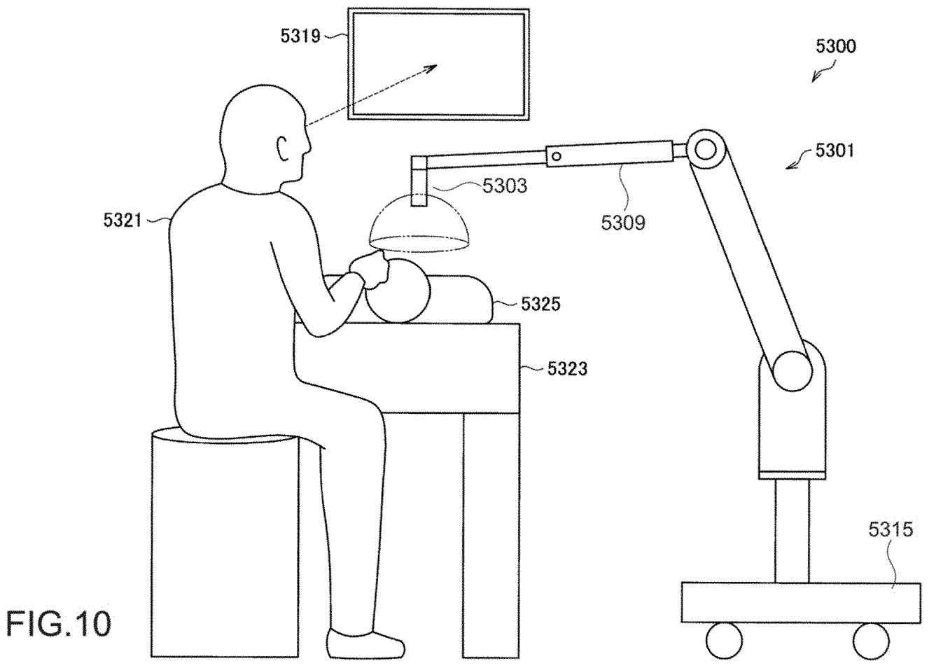

FIG. 10 is a view depicting an example of a schematic configuration of a microscopic surgery system 5300 according to another embodiment. In FIG. 10, a state is schematically illustrated in which a surgeon 5321 is using the microscopic surgery system 5300 to perform surgery for a patient 5325 on a patient bed 5323.

The microscope apparatus 5301 has a microscope unit 5303 for enlarging an observation target (surgical region of a patient) for observation, an arm unit 5309 which supports the microscope unit 5303 at a distal end thereof, and a base unit 5315 which supports a proximal end of the arm unit 5309.

Further, light for illumination is provided to the microscope apparatus 5301 from the medical observation system 100 according to the present technology. For example, the light source unit 10, the relay optical system 30, and the like are provided inside or in the vicinity of the base unit 5315. The outer light guide 31 is, for example, inserted to the microscope unit 5303 along the arm unit 5309. It is to be noted that the medical observation system 100 may be provided in another casing.

As depicted in FIG. 10, upon surgery, using the microscopic surgery system 5300, an image of a surgical region picked up by the microscope apparatus 5301 is displayed in an enlarged scale on the display apparatus 5319 installed on a wall face of the surgery room. The display apparatus 5319 is installed at a position opposing to the surgeon 5321, and the surgeon 5321 would perform various treatments for the surgical region such as, for example, resection of the affected area while observing a state of the surgical region from a video displayed on the display apparatus 5319.

The light emitted from the outer light guide 31 is irradiated from the illumination optical system 32 provided in the microscope unit 5303 toward an operating field. Thus, it is possible to irradiate the operating field with, for example, bright white light or the like that has small color unevenness and shoot a high-quality surgical operation image or the like.

Among the characteristic parts according to the present technology described above, it is also possible to combine at least two characteristic parts together. That is, the various characteristic parts described in the respective embodiments may be arbitrarily combined together without being distinguished from each other between the respective embodiments. Further, the various effects described above are given only for illustration and should not be interpreted in a limited way. Further, other effects may be produced.

Note that the present technology may also employ the following configurations.
(1) A medical observation system including:
   a light source having a plurality of light-emitting elements, each of which emits light;
   an optical member arranged to reflect the light emitted from the plurality of light-emitting elements and make the reflected light incident on a first region;
   a first light guide body that is arranged in the first region, has an incident end and an emission end on a side opposite to the incident end, and guides the light incident from the incident end to the emission end; and
   an imaging element that irradiates an operating field with the guided light and captures an image of light reflected by a subject.
(2) The medical observation system according to (1), in which
   the first light guide body uniformizes brightness distribution at the emission end of the light emitted from the emission end.
(3) The medical observation system according to (1) or (2), in which
   the plurality of light-emitting elements is arranged around a prescribed axis, and
   the optical member has a first reflection unit that is arranged facing the plurality of light-emitting elements and reflects the light emitted from the plurality of light-emitting elements to be condensed toward a second region on the prescribed axis.
(4) The medical observation system according to (3), in which
   the plurality of light-emitting elements emits the light parallel to the prescribed axis.
(5) The medical observation system according to (3) or (4), in which
   the first reflection unit includes at least one of a parabolic mirror or a free-form surface mirror.
(6) The medical observation system according to (5), in which
   the free-form surface mirror includes a plurality of divided mirrors.
(7) The medical observation system according to any one of (3) to (6), in which
   the second region is the first region.
(8) The medical observation system according to any one of (3) to (6), in which
   the optical member has a second reflection unit that is arranged facing the first reflection unit and reflects the light toward the first region, the light being directed from the first reflection unit to the second region.
(9) The medical observation system according to (8), in which
   the second reflection unit includes at least one of a parabolic mirror, a plane mirror, or a free-form surface mirror.
(10) The medical observation system according to any one of (1) to (9), in which
   the plurality of light-emitting elements includes a plurality of types of light-emitting elements that emits light of different wavelength ranges.
(11) The medical observation system according to any one of (1) to (10), in which
   the plurality of light-emitting elements includes at least one of a light-emitting element that emits red light, a light-emitting element that emits green light, or a light-emitting element that emits blue light.
(12) The medical observation system according to any one of (1) to (11), in which
   the plurality of light-emitting elements includes at least one of a light-emitting element that emits infrared light or a light-emitting element that emits ultraviolet light.
(13) The medical observation system according to any one of (1) to (12), in which
   the plurality of light-emitting elements is arranged such that an incident angle of the light with respect to the incident end falls within a prescribed range, the light being emitted from at least one of the same type of light-emitting elements.
(14) The medical observation system according to any one of (1) to (13), in which
   the plurality of light-emitting elements includes laser diodes.
(15) The medical observation system according to any one of (1) to (14), in which
   the plurality of light-emitting elements is arranged on the same radiation plate.
(16) The medical observation system according to any one of (1) to (15), further including:
   a second light guide body that guides the light to an observation target; and
   a relay optical system that connects the light emitted from the emission end of the first light guide body to an incident end of the second light guide body.
(17) The medical observation system according to (16), in which
   an area of the emission end of the first light guide body is smaller than an area of the incident end of the second light guide body.
(18) The medical observation system according to any one of (1) to (17), in which
   the medical observation system is constituted as a microscopic system or an endoscopic system.
(19) The medical observation system according to any one of (1) to (18), in which
   the optical member includes a lens unit that condenses the light on the first region, the light being reflected toward the first region.
(20) The medical observation system according to any one of (1) to (19), in which
   the incident end is arranged in the first region.
(21) The medical observation system according to (20), in which
   the optical member has a reflection plate arranged to reflect the light emitted from the plurality of light-emitting elements and make the reflected light condensed on the incident end.
(22) The medical observation system according to (21), in which
   the reflection plate is arranged so that respective spots of the light emitted from the plurality of light-emitting elements overlap each other on an end surface of the incident end.
(23) The medical observation system according to (21) or (22), in which
   the reflection plate includes one of a parabolic mirror and a free-form surface mirror.
(24) The medical observation system according to any one of (1) to (23), in which
   the optical member is arranged to reflect the light emitted from the plurality of light-emitting elements and make the respective light axes of the reflected light incident on the incident end from different directions.
(25) The medical observation system according to any one of (1) to (24), in which
   the optical member includes one or more optical elements and is arranged so that respective light emitted from the plurality of light-emitting elements passes through the same number of the optical elements.
(26) The medical observation system according to any one of (1) to (27), in which
   the first light guide body has a light guide axis passing through the incident end and the emission end, and
   the plurality of light-emitting elements is arranged around the light guide axis.
(27) A medical light source apparatus including:
   a light source having a plurality of light-emitting elements, each of which emits light;
   an optical member arranged to reflect the light emitted from the plurality of light-emitting elements and make the reflected light incident on a prescribed region; and
   a light guide body that is arranged in the prescribed region, has an incident end and an emission end on a side opposite to the incident end, and guides the light incident from the incident end to the emission end.
(28) A medical illumination method including:
   causing each of a plurality of light-emitting elements to emit light;
   reflecting the light emitted from the plurality of light-emitting elements and making the reflected light incident on a prescribed region; and
   guiding the light incident from the incident end to the emission end by a light guide body that is arranged in the prescribed region and has the incident end and an emission end on a side opposite to the incident end.
(29) A medical light source apparatus including:
   light-emitting means having a plurality of light-emitting elements, each of which emits light;
   condensing means arranged to reflect the light emitted from the plurality of light-emitting elements and make the reflected light incident on a prescribed region; and
   light guide means that is arranged in the prescribed region, has an incident end and an emission end on a side opposite to the incident end, and guides the light incident from the incident end to the emission end.

### REFERENCE SIGNS LIST

3 light axis
4 condensing region
5 focus region
10, 110, 210, 310, 410, 510 light source unit
11, 111, 211, 311, 411, 511 light source
12, 112, 212, 312, 412, 512 optical member
13, 113, 213, 313, 413, 513 inner light guide
15, 15R, 15G, 15B, 15IR, 15UV laser diode
18, 118, 218, 318, 418, 518 incident end
20, 320 emission end
30 relay optical system
31 outer light guide
40 imaging element
50, 150, 250, 550 reflector
350, 450 first reflector
370, 470 second reflector
460 lens unit
100 medical observation system

1. A medical observation system comprising:
a light source having a plurality of light-emitting elements, each of which emits light;
an optical member arranged to reflect the light emitted from the plurality of light-emitting elements and make the reflected light incident on a first region;
a first light guide body that is arranged in the first region, has an incident end and an emission end on a side opposite to the incident end, and guides the light incident from the incident end to the emission end; and
an imaging element that irradiates an operating field with the guided light and captures an image of light reflected by a subject.

2. The medical observation system according to claim 1, wherein
the first light guide body uniformizes brightness distribution at the emission end of the light emitted from the emission end.

3. The medical observation system according to claim 1, wherein
the plurality of light-emitting elements is arranged around a prescribed axis, and
the optical member has a first reflection unit that is arranged facing the plurality of light-emitting elements and reflects the light emitted from the plurality of light-emitting elements to be condensed toward a second region on the prescribed axis.

4. The medical observation system according to claim 3, wherein
the plurality of light-emitting elements emits the light parallel to the prescribed axis.

5. The medical observation system according to claim 3, wherein
the first reflection unit includes at least one of a parabolic mirror or a free-form surface mirror.

6. The medical observation system according to claim 5, wherein
the free-form surface mirror includes a plurality of divided mirrors.

7. The medical observation system according to claim 3, wherein
the second region is the first region.

8. The medical observation system according to claim 3, wherein
the optical member has a second reflection unit that is arranged facing the first reflection unit and reflects the light toward the first region, the light being directed from the first reflection unit to the second region.

9. The medical observation system according to claim 8, wherein
the second reflection unit includes at least one of a parabolic mirror, a plane mirror, or a free-form surface mirror.

10. The medical observation system according to claim 1, wherein
the plurality of light-emitting elements includes a plurality of types of light-emitting elements that emits light of different wavelength ranges.

11. The medical observation system according to claim 1, wherein
the plurality of light-emitting elements includes at least one of a light-emitting element that emits red light, a light-emitting element that emits green light, or a light-emitting element that emits blue light.

12. The medical observation system according to claim 1, wherein
the plurality of light-emitting elements includes at least one of a light-emitting element that emits infrared light or a light-emitting element that emits ultraviolet light.

13. The medical observation system according to claim 1, wherein
the plurality of light-emitting elements is arranged such that an incident angle of the light with respect to the incident end falls within a prescribed range, the light being emitted from at least one of the same type of light-emitting elements.

14. The medical observation system according to claim 1, wherein
the plurality of light-emitting elements includes laser diodes.

15. The medical observation system according to claim 1, wherein
the plurality of light-emitting elements is arranged on the same radiation plate.

16. The medical observation system according to claim 1, further comprising:
a second light guide body that guides the light to an observation target; and
a relay optical system that connects the light emitted from the emission end of the first light guide body to an incident end of the second light guide body.

17. The medical observation system according to claim 16, wherein
an area of the emission end of the first light guide body is smaller than an area of the incident end of the second light guide body.

18. The medical observation system according to claim 1, wherein
the medical observation system is constituted as a microscopic system or an endoscopic system.

19. A medical light source apparatus comprising:
a light source having a plurality of light-emitting elements, each of which emits light;
an optical member arranged to reflect the light emitted from the plurality of light-emitting elements and make the reflected light incident on a prescribed region; and
a light guide body that is arranged in the prescribed region, has an incident end and an emission end on a side opposite to the incident end, and guides the light incident from the incident end to the emission end.

20. A medical illumination method comprising:
causing each of a plurality of light-emitting elements to emit light;
reflecting the light emitted from the plurality of light-emitting elements and making the reflected light incident on a prescribed region; and
guiding the light incident from the incident end to the emission end by a light guide body that is arranged in the prescribed region and has the incident end and an emission end on a side opposite to the incident end.
